(19) United States
(12) Patent Application Publication
(10) Pub. No.: US 2022/0206167 A1
(43) Pub. Date: Jun. 30, 2022

(54) Blast Movement Monitor, System and Method

(71) Applicant: Leica Geosystems Pty Ltd, Brisbane City, Queensland (AU)

(72) Inventors: Kausik DASGUPTA, Sumner, Queensland (AU); Jacques JANSE VAN RENSBURG, Sumner, Queensland (AU)

(21) Appl. No.: 17/612,425

(22) PCT Filed: Jun. 26, 2020
(86) PCT No.: PCT/AU2020/050668

§ 371 Date: Nov. 18, 2021

(30) Foreign Application Priority Data
Jun. 28, 2019 (AU) 2019902277

(51) Int. Cl. G01S 19/49; F42D 3/04; E21C 37/00

(52) U.S. Cl. CPC G01S 19/49; H04W 84/18; E21C 37/00; F42D 3/04

(57) ABSTRACT

The invention relates to a method of monitoring the movement of an ore body resulting from blasting, the method comprising: positioning a plurality of blast movement monitors in a blast zone in the ore body, each of the blast movement monitors having a monitor identifier; attributing pre-blast coordinates to said blast movement monitors; blasting the ore body; attributing post-blast coordinates to said blast movement monitors; collating said post-blast coordinates and transmitting said post-blast coordinates to a data collector, wherein post-blasting said blast movement monitors form a sub-surface mesh network and said step of collating said post-blast coordinates comprises communicating said post-blast coordinates between blast movement monitors within said sub-surface mesh network.

### BACKGROUND

The disclosed embodiments relate to blast movement monitors, systems and methods for determining the movement of an ore body during blasting operations. In particular, the embodiments relate to blast movement monitors that are adapted to transmit location data to one another post blasting operations in order to identify post blasting positioning of the blast movement monitors and transmit this data to a data collector for subsequent analysis. Similarly, the disclosed methods involve the transmission of location data between a plurality of blast movement monitors post blasting operations and collation of this data for transmission to a data collector.

This embodiments relate particularly but not exclusively to methods of determining the movement of an ore boundary. Typically the boundary might be between high grade ore, e.g. a vein of gold ore, and a low grade ore, in a heterogeneous ore body of an open cast mine that practises open cut selective mining. It will therefore be convenient to hereinafter describe the disclosed invention with reference to this example application. However it is to be clearly understood that the invention is capable of broader application. For example the inventive embodiments may be used to determine the movement in boundaries between ore and waste for many ores. It may also be used to measure the boundary movement between sulphide ore and oxide ore in fractional deposits. These ores require different concentration processes and therefore need to be recovered separately. It may also be used to measure the movement of the edge of a coal seam when the overburden is blasted.

Open cut mining operations are well known and are conducted in a number of countries around the world. Typically they comprise progressively mining domains of an ore body in a staged batch-like process. Each so called batch comprises selectively placing explosives in the rock of the batch. Thereafter the rock is blasted to break and loosen the rock and form a muck pile. Typically the deposits in these mines are heterogeneous in the sense that the ore is disseminated in complex shaped volumes of varying grade within a host rock which is waste. The shape of each ore zone on a horizontal plane is represented by a polygon when viewed in plan.

The rock body for example might comprise one or more ore polygons that are economic to recover and waste rock that is to be discarded. The ore is selectively removed from the muck pile and sent to a concentrator where the valuable mineral is extracted by an appropriate technique. Similarly the waste rock is removed and sent to a discard rock dump. Clearly an important part of this process is the accurate delineation of and identification of the boundaries between high grade ore and low grade ore and between ore and waste. A mixture of scientific know how, geology, computer algorithms, and experience is used to determine the boundaries in the body of rock prior to blasting being conducted. This art has developed to the point where mining engineers and geologists have a good three dimensional picture of the boundaries between the different ores in the virgin rock prior to blasting.

However, it is quite clear that the rock moves when it is subjected to blasting. The blasting causes some expansion of the rock and in addition there may be some differences in the amount of movement of the different parts of the rock. This is illustrated schematically in FIG. 1. Historically, mining engineers and geologists sometimes worked on the assumption that the ore boundaries of the blasted rock are the same as that for the unblasted rock and directed the broken rock to respectively the concentrator and the dump on this basis. More recently movement of blasted rock has been determined using blast monitors that are positioned within the rock body prior to blasting and their location identified post blast. This is generally achieved by including a transmitter in each blast monitor that transmits a signal that is detected by an external detector post blasting operations.

The systems currently available in the market, and briefly discussed above, require a user to detect transmitters post blast by walking on the muck-pile, creating the opportunity for accidental trips and falls in a hazardous environment. This is not ideal as sites continuously look towards identifying safer work practices.

The subject matter claimed herein is not limited to embodiments that solve any disadvantages or that operate only in environments such as those described above. Rather, this background is only provided to illustrate exemplary technology areas where some embodiments described herein may be practiced.

### SUMMARY

As mentioned above, the disclosed embodiments relate generally to blast movement monitors, systems and methods for determining the movement of an ore body during blasting operations.

According to one aspect there is provided a method of monitoring the movement of an ore body resulting from blasting, the method comprising:

positioning a plurality of blast movement monitors in a blast zone in the ore body, each of the blast movement monitors having a monitor identifier;

attributing pre-blast coordinates to the blast movement monitors;

blasting the ore body;

attributing post-blast coordinates to the blast movement monitors;

collating the post-blast coordinates and transmitting the post-blast coordinates to a data collector,

wherein post-blasting the blast movement monitors form a sub-surface mesh network and the step of collating the post-blast coordinates comprises communicating the post-blast coordinates between blast movement monitors within the sub-surface mesh network.

Positioning of the blast movement monitors generally comprises locating the blast movement monitors in an array of holes in the ore body. Positioning may be somewhat dependent on the blasting strategy and the desired monitoring outcome. For example, several monitors may be located in one or more holes. In certain embodiment, each of the blast movement monitors is located in a dedicated hole in the ore body.

According to preferred embodiments each of the blast movement monitors has a unique monitor identifier. For example, each blast movement monitor may be allocated a unique radio-frequency identification (RFID). In certain embodiments it is envisaged that each blast movement monitor communicate a unique monitor identifier using modulation.

Attributing pre-blast coordinates to the blast movement monitors may comprise pre-programming high precision GNSS coordinates to each of the blast movement monitors. For example, this may be achieved with a user device (e.g. a hand-held device), for example on placement of the blast movement monitors in the holes in the ore body. It is envisaged that attributing pre-blast coordinates to the blast movement monitors may also comprise positioning the blast movement monitors in the ore body and transmitting pre-blast coordinates from the positioned blast movement monitors to the data collector. For example, the blast movement monitors may calculate their respective pre-blast coordinates and transmit these to the data collector. In an alternative embodiment, monitor identifiers and pre-blast coordinates of each of the blast movement monitors are recorded on the user device. In this embodiment, after recording the pre-blast coordinates of the blast movement monitors on the user device, internal coordinates of the blast movement monitors may be zeroed prior to blasting the ore body. It is considered this embodiment may provide advantages in that data storage on the individual blast movement monitors is minimised, thereby simplifying the design of the blast movement monitors.

Attributing post-blast coordinates to the blast movement monitors may comprise each of the blast movement monitors calculating its post-blast coordinate. In that regard, the internal electronics of the blast movement monitors preferably include an inertial measurement unit (IMU), such as gyroscopes, accelerometers, and magnetometers, and a CPU. According to this embodiment, the CPU calculates the displacement of the blast movement monitor during blasting using inputs from the IMU. This must be achieved in the post-blast environment, which is generally a GPS denied environment.

Collating of the post-blast coordinates may be initiated by a transmission request from the data collector. For example, the transmission request may be transmitted at least 10 minutes post-blast, preferably at least 15 minutes post-blast. This allows for the sub-surface blasted material to come to rest post-blasting operations.

The blast movement monitors within a transmission distance from one another in the sub-surface mesh network communicate their respective post-blast coordinates to one another until all post-blast coordinates are preferably collated in a final one of the blast movement monitors in closest proximity to the data collector. In effect, the blast movement monitors ‘talk’ to each other to communicate the post-blast coordinates within the sub-surface mesh network until the post-blast coordinates are finally collated as a data set.

The data collector may be located at any suitable location. It may be positioned pre-blast or post-blast. Generally, the data collector is located on a surface at the perimeter of the blast zone.

According to another aspect there is provided a system for monitoring the movement of an ore body resulting from blasting, the system comprising:

a plurality of blast movement monitors, each of the blast movement monitors having a monitor identifier; and

a data collector,

wherein the blast movement monitors are adapted to communicate respective post-blast coordinates within a sub-surface mesh network formed by the blast movement monitors post-blasting and collate the post-blast coordinates for transmission to the data collector.

Once again, each of the blast movement monitors preferably has a unique monitor identifier. The blast movement monitors may be adapted to be pre-programmed with high precision GNSS pre-blast coordinates, or may be adapted to self-identify respective pre-blast coordinates. In an alternative embodiment, the system further comprises a user device, wherein the monitor identifiers and pre-blast coordinates of each of the blast movement monitors are recorded on the user device. In this embodiment, internal coordinates of the blast movement monitors can be zeroed prior to blasting the ore body.

The blast movement monitors are preferably adapted to calculate their respective post-blast coordinates as described above, for example using inputs from an internal IMU, including gyroscope, accelerometer and magnetometer components, or combinations thereof. Blast movement monitors within a transmission distance from one another in the sub-surface mesh network may be adapted to communicate their respective post-blast coordinates to one another until all post-blast coordinates are preferably collated in a final one of the blast movement monitors in closest proximity to the data collector.

Again, the data collector may be located on a surface at the perimeter of the blast zone. The system may also further comprise one or more data monitors adapted to receive the post-blast coordinates from the blast movement monitors and transmit same to the data collector.

According to another aspect there is provided a blast movement monitor for monitoring the movement of an ore body resulting from blasting, the blast movement monitor comprising:

a housing having an internal space;

electronic circuitry disposed within the internal space and comprising a central processing unit (CPU), an inertial measurement unit (IMU), and a transmitter and receiver; and

a power supply associated with the electronic circuitry,

wherein the central processing unit (CPU) is adapted to calculate a post-blast coordinate of the blast movement monitor using inputs from the inertial measurement unit (IMU).

The housing preferably comprises an internal mounting portion that defines the internal space and is adapted to mount said electronic circuitry, a base portion and a cooperating cap portion adapted to engage with the base portion, whereby the base portion and cap portion encapsulate the internal mounting portion. Accordingly, the electronic circuitry may be mounted in the internal mounting portion and the internal mounting portion engaged or secured in the base portion. This may include engagement with a locking mechanism and/or gluing of the internal mounting portion in the base portion. The cap portion may then be coupled with the base portion to securely encapsulate the internal mounting portion and the electronic circuitry.

In certain embodiments, the electronic circuitry is disposed on a displacement sensor board. According to this embodiment, the displacement sensor board is mounted on the internal mounting portion of the housing. The displacement sensor board may be coupled with a mother board adapted to store data received from the electronic circuitry.

As previously described, the central processing unit (CPU) is adapted to calculate a post-blast coordinate of the blast movement monitor using inputs from the inertial measurement unit (IMU). The inertial measurement unit (IMU) may, for example, comprises a gyroscope, accelerometer, magnetometer (e.g. 3-axis magnetometer) or any combination of one or more thereof.

The transmitter and receiver are preferably adapted to communicate post-blast coordinates with other blast movement monitors within proximity after blasting operations. The transmitter and receiver may be selected from a Bluetooth transmitter and receiver, radio transmitter and receiver, WiFi transmitter and receiver or any combination thereof. It is considered, however, that the transmitter and receiver will preferably include low frequency communication protocols.

The blast movement monitor includes a power supply that powers the electronic circuitry. Preferably, the power supply comprises a battery. In that regard, it is considered that it may be advantageous if the blast movement monitor can operate in a ‘sleep’ mode so that charge is maintained. As such, the blast movement monitor is preferably adapted to be activated remotely or on blasting. For example, blasting may ‘wake’ the electronic circuitry, or this may be achieved by an external signal.

### BRIEF DESCRIPTION OF THE DRAWINGS

To further clarify various aspects of some embodiments of the present invention, a more particular description will be rendered by references to specific embodiments, which are illustrated in the appended drawings. It should be appreciated that these drawings depict only typical embodiments of the invention and are therefore not to be considered limiting on its scope. In the accompanying drawings:

FIG. 1 illustrates schematically in plan view a likely movement of a rock body as a result of blasting.

FIG. 2 illustrates data flow between blast movement monitors post-blast.

FIG. 3 illustrates a cross section of a housing for a blast movement monitor according to the disclosure.

FIG. 4 illustrates a top view of the housing of FIG. 3.

FIG. 5 illustrates the housing of FIG. 3 in a closed orientation.

FIGS. 6 and 7 illustrate a mounting arrangement for mounting electronics within the housing according to an embodiment of the disclosure.

FIG. 8 illustrates a top view of a displacement sensor board according to an embodiment respectively.

FIG. 9 illustrates a bottom view of the displacement sensor board of FIG. 8.

FIG. 10 illustrates a schematic top view of the displacement sensor board of FIGS. 8 and 9.

FIG. 11 illustrates a schematic bottom view of the displacement sensor board of FIGS. 8 and 9.

### DETAILED DESCRIPTION OF THE PREFERRED EMBODIMENTS

Hereinafter, this specification will describe the present invention according to the preferred embodiments. It is to be understood that limiting the description to the preferred embodiments of the invention is merely to facilitate discussion of the present invention and it is envisioned without departing from the scope of the appended claims.

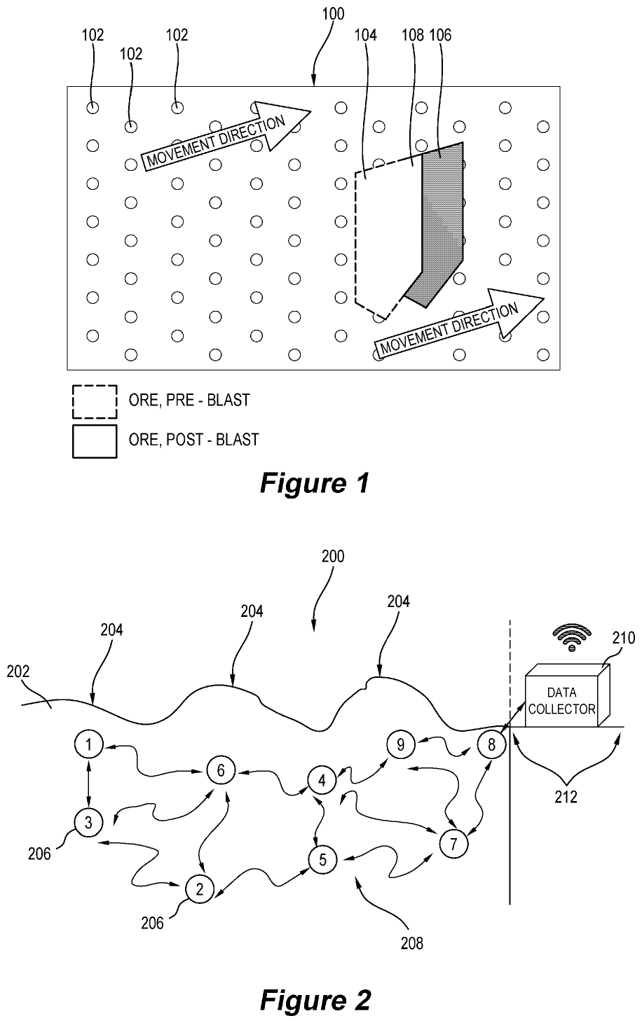

Referring to FIG. 1, briefly, during blasting operations of an ore body 100, a number of holes 102 are drilled in the ore body 100 and explosives placed in the holes 102. Without using blast movement monitors a mine site may not know that the pre-blast ore location 104 has translated to a post-blast ore location 106 after a blast, which may lead to dilution and ore loss and the recovery of only a recovery portion 108 of the blasted ore.

Referring to FIG. 2, the data flow of a system 200 for monitoring the movement of an ore body 202 resulting from blasting is illustrated. As illustrated, the post-blast surface 204 of the muck pile is generally rough and difficult to navigate. The post-blast surface 204 is therefore hazardous to workers who are tasked with identifying where blast movement monitors are located post-blast, which has in the past been achieved with a handheld device.

The system 200 illustrated includes a plurality of blast movement monitors 206 that are buried under the post-blast surface 204 of the muck pile. The blast movement monitors 206 form a sub-surface mesh network 208. The arrows depict data flow between blast movement monitors 206 within the sub-surface mesh network 208 buried under the surface 204. The data includes post-blast coordinates for each of the blast movement monitors 206.

Data moves between the blast movement monitors 206 and is collated in a final blast movement monitor 206′ of the blast movement monitors 206. Each blast movement monitors 206 has a unique ID which is used for identification during data collection. The collated data is transmitted to a data collector 210 by the final blast movement monitor 206′.

In one embodiment, the blast movement monitors 206 are pre-programmed with high precision GNSS coordinates (x, y, z) before a blast. This is done using a hand-held device (not shown) when being placed in a hole in the blast area. The blast movement monitors 206 are then placed allowed to come to rest in their pre-blast positions. The data collector 210 is placed on the surface of the closest non-blast area 212 to the perimeter of the blast zone or could be taken to the post blast area after the blast.

Once all blast movement monitors 206 are placed in the blast area and allowed to settle, the data collector 210 sends out a transmission requesting the pre-blast locations of all blast movement monitors 206. The nearest blast movement monitors 206′ responds with x, y, z values of all blast movement monitors 206, identified by their respective unique IDs, in the blast.

In another embodiment, the unique ID of the blast movement monitors 206 are recorded using a hand-held device along with the GNSS coordinates (X, Y, Z) of the install point. The depth of install is also measured and recorded in the hand-held device. The internal coordinates (x, y, z) of the blast movement monitors 206 are zeroed (0, 0, 0) before a blast. The blast movement monitors 206 are then placed in their pre-blast positions.

Once the blast occurs, the blast movement monitors 206 move with the subsurface material and come to rest within 10 minutes of the blast occurring. All blast movement monitors 206 calculate their displacement in all three axes (x1, y1, z1) and transmit the data to all blast movement monitors 206 within range as shown in FIG. 2. About 15 minutes after the blast, the data collector 210 sends out a transmission requesting the final positions of all blast movement monitors 206. The nearest blast movement monitors 206′ responds with x1, y1, z1 values of all blast movement monitors 206 (identified by their respective unique IDs) in the blast. The data collector calculates the final resting locations of blast movement monitors 206 using the displacement values received from blast movement monitors 206 and the GNSS Coordinates (X, Y, Z) of the install locations of blast movement monitors 206 recorded using the hand-held device at the time of install.

If Wi-Fi is available in the mine pit, the data gathered by the data collector 210 can be made available at the mine offices of the geologists within seconds of acquiring it. The data collector 210 is the removed from the location and stored for use in the next blast. The data collector 210 may also have the option of transferring data using a cable.

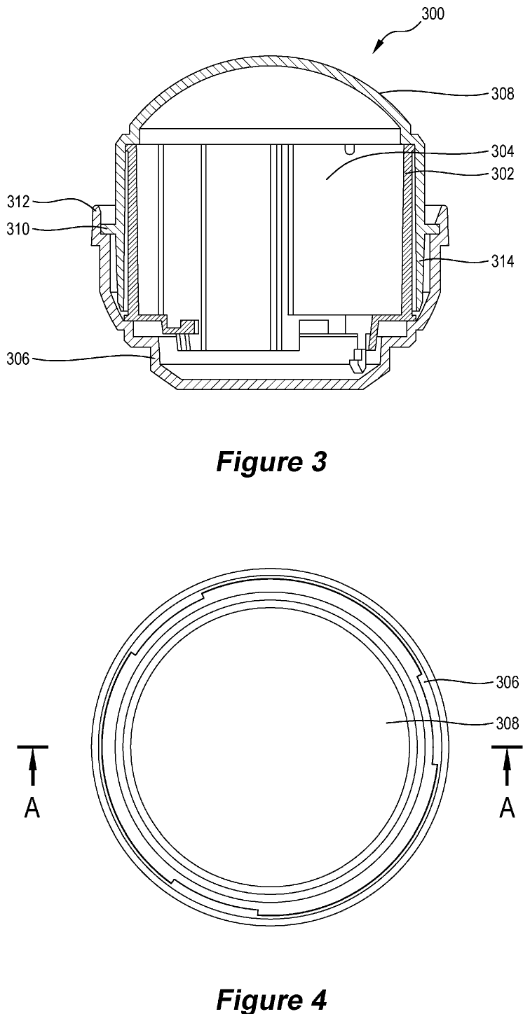

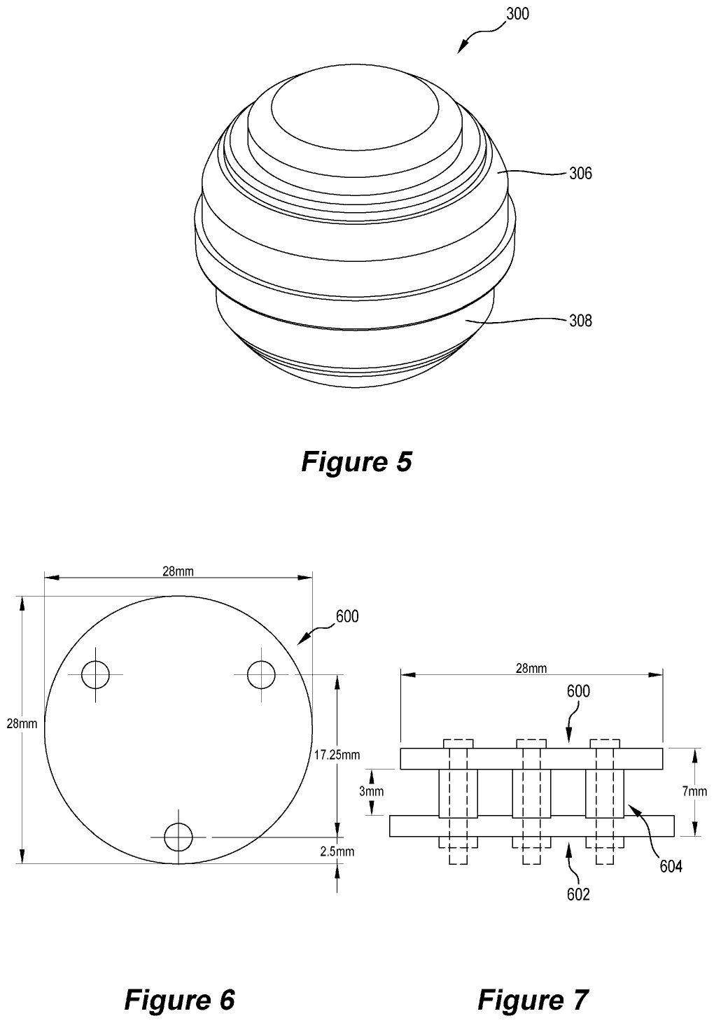

Turning to FIGS. 3 to 5, a housing 300 of a blast movement monitor is illustrated. The housing 300 comprises an internal mounting portion 302 that defines an internal space 304. The internal mounting portion 302 is adapted to mount electronic circuitry of the blast movement monitor. The housing further comprises a base portion 306 and a cooperating cap portion 308 adapted to engage with the base portion 306. The base portion 306 and cap portion 308 therefore encapsulate the internal mounting portion 302 when they are engaged with each other. As illustrated, the cap portion 308 includes a ridge 310 that couples with a collar 312 of the base portion 306 when the cap portion 306 and base portion 308 are engaged. The circumferential wall 314 of the cap portion 306 extends into the base portion 308 past the ridge 310.

Referring to FIGS. 6 and 7, a displacement sensor board 600 is illustrated. The displacement sensor board 600 includes the electronic circuitry of the blast movement monitor, as will be discussed in more detail below, and is adapted to be mounted in the interior space 304 of the internal housing portion 302 of the housing 300. The displacement sensor board 600 is coupled with a mother board 602 with spacers 604. It is further envisaged that the electronic circuitry may be fitted to one circuit board and such an embodiment is considered within the scope of the present invention. The invention should not be considered bound to the embodiment illustrated.

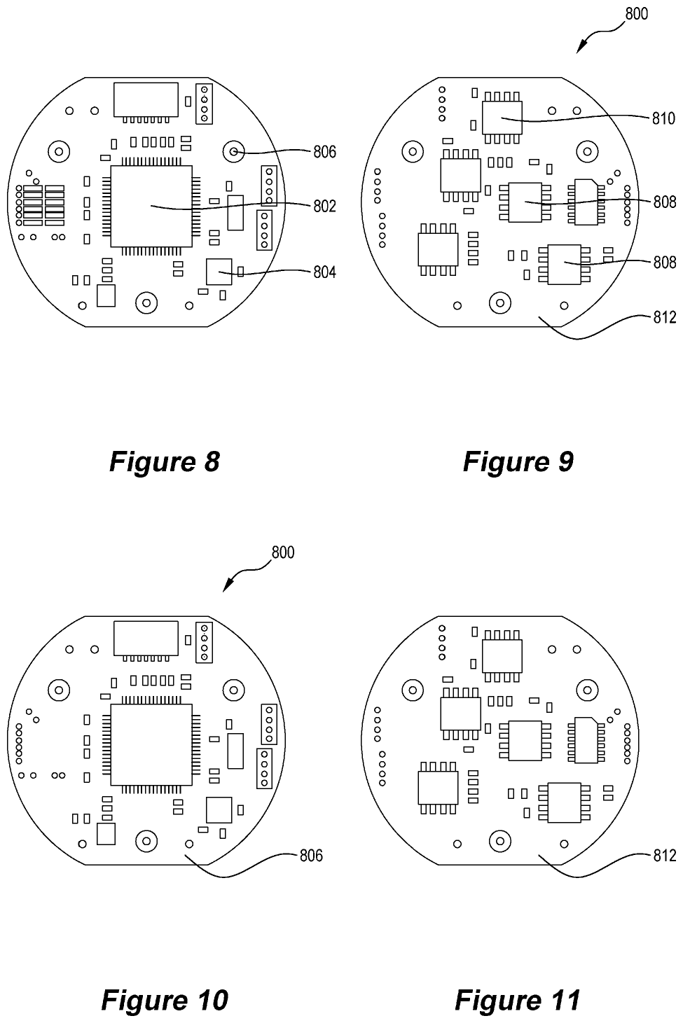

FIGS. 8 to 11 provide more detail of the electronic circuitry on a displacement sensor board 800. As illustrated the displacement sensor board 800 includes, amongst other components, a CPU 802, an inertial measurement unit (IMU) 804 on a top surface 806 of the displacement sensor board 800. Power supply regulators 808 and a logic level translator 810 are included on a bottom surface 812 of the displacement sensor board 800. Once again, it should be clearly noted that the electronic circuitry illustrated is only one example of a possible embodiment of the invention. It is envisaged that many other options may be suitable and within the broad ambit of the invention.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of steps, elements or integers. Thus, in the context of this specification, the term “comprising” is used in an inclusive sense and thus should be understood as meaning “including principally, but not necessarily solely”.

Unless the context requires otherwise or specifically stated to the contrary, integers, steps or elements recited herein as singular integers, steps or elements clearly encompass both singular and plural forms of the recited integers, steps or elements.

It will be appreciated that the foregoing description has been given by way of illustrative example of the invention and that all such modifications and variations thereto as would be apparent to persons of skill in the art are deemed to fall within the broad scope and ambit of the invention as herein set forth.

1. A method of monitoring movement of an ore body resulting from blasting, comprising:
positioning a plurality of blast movement monitors in a blast zone in the ore body, each of said blast movement monitors having a monitor identifier;
attributing pre-blast coordinates (x, y, z) to said blast movement monitors;
blasting the ore body;
attributing post-blast coordinates (x′, y′, z′) to said blast movement monitors;
collating said post-blast coordinates (x′, y′, z′) and transmitting said post-blast coordinates (x′, y′, z′) to a data collector, wherein
after said blasting, said blast movement monitors form a sub-surface mesh network and each of said blast movement monitors calculates its post-blast coordinates (x′, y′, z′), and
said step of collating said post-blast coordinates (x′, y′, z′) comprises communicating said post-blast coordinates (x′, y′, z′) between blast movement monitors within said sub-surface mesh network.

2-3. (canceled)

4. The method according to claim 1, wherein each of said blast movement monitors has a unique monitor identifier.

5. The method according to claim 1, wherein the step of attributing pre-blast coordinates to said blast movement monitors comprises pre-programming high precision GNSS coordinates to each of said blast movement monitors, or positioning the blast movement monitors in the ore body and transmitting pre-blast coordinates from the positioned blast movement monitors to the data collector.

6. (canceled)

7. The method according to claim 1, wherein monitor identifiers and pre-blast coordinates of each of the blast movement monitors are recorded on a user device.

8. The method according to claim 7, wherein after the pre-blast coordinates of the blast movement monitors are recorded on the user device, internal coordinates of the blast movement monitors are zeroed prior to blasting the ore body.

9. The method according to claim 1, wherein each of said blast movement monitors calculates its post-blast coordinate using inputs from an inertial measurement unit (IMU) and a magnetometer.

10. The method according to claim 1, wherein collating of said post-blast coordinates is initiated by a transmission request from said data collector.

11. (canceled)

12. The method according to claim 1, wherein blast movement monitors within a transmission distance from one another in said sub-surface mesh network communicate their respective post-blast coordinates to one another until all post-blast coordinates are collated in a final one of said blast movement monitors in closest proximity to said data collector.

13. (canceled)

14. A system for monitoring movement of an ore body resulting from blasting, comprising:
a plurality of blast movement monitors, each of said blast movement monitors having a monitor identifier; and
a data collector,
wherein each of said blast movement monitors is adapted to calculate its post-blast coordinates (x′, y′, z′) and communicate respective post-blast coordinates (x′, y′, z′) within a sub-surface mesh network formed by said blast movement monitors after blasting until the post-blast coordinates (x′, y′, z′) are collated for transmission to said data collector.

15. The system according to claim 14, wherein each of said blast movement monitors has a unique monitor identifier.

16. The system according to claim 14, wherein said blast movement monitors are adapted to be pre-programmed with high precision GNSS pre-blast coordinates, or wherein said blast movement monitors are adapted to self-identify respective pre-blast coordinates.

17. (canceled)

18. The system according to claim 14, further comprising a user device, wherein the monitor identifiers and pre-blast coordinates of each of the blast movement monitors are recorded on the user device.

19. The system according to claim 18, wherein internal coordinates of the blast movement monitors can be zeroed prior to blasting the ore body.

20. The system according to claim 14, wherein each of said blast movement monitors calculates its post-blast coordinate using inputs from an inertial measurement unit (IMU) and a magnetometer.

21. The system according to claim 14, wherein blast movement monitors within a transmission distance from one another in said sub-surface mesh network are adapted to communicate their respective post-blast coordinates to one another until all post-blast coordinates are collated in a final one of said blast movement monitors in closest proximity to said data collector.

22. (canceled)

23. A blast movement monitor for monitoring movement of an ore body resulting from blasting, comprising:
a housing having an internal space;
electronic circuitry disposed within the internal space and comprising a central processing unit (CPU), an inertial measurement unit (IMU), and a transmitter and receiver; and
a power supply associated with said electronic circuitry, wherein
said central processing unit (CPU) is adapted to calculate a post-blast coordinate (x′, y′, z′) of said blast movement monitor using inputs from said inertial measurement unit (IMU) and communicate the post-blast coordinates (x′, y′, z′) to the transmitter, and
the transmitter and receiver are adapted to communicate post-blast coordinates (x′, y′, z′) with other like blast movement monitors within a sub-surface mesh network formed by the blast movement monitors after blasting.

24. The blast movement monitor according to claim 23, wherein said housing comprises an internal mounting portion that defines said internal space and is adapted to mount said electronic circuitry, a base portion and a cooperating cap portion adapted to engage with said base portion, whereby said base portion and cap portion encapsulate said internal mounting portion.

25. The blast movement monitor according to claim 23, wherein said electronic circuitry is disposed on a displacement sensor board mounted on said internal mounting portion of said housing.

26-27. (canceled)

28. The blast movement monitor according to claim 23, wherein said inertial measurement unit (IMU) comprises one or more of a gyroscope, an accelerometer, and a 3-axis magnetometer alone or in combination.

29. The blast movement monitor according to claim 23, wherein said transmitter and receiver use low frequency communication protocols.

30. (canceled)

31. The blast movement monitor according to claim 23, wherein said blast movement monitor is adapted to be activated remotely or on blasting.
